(19) United States
(12) Patent Application Publication
(10) Pub. No.: US 2009/0162415 A1
(43) Pub. Date: Jun. 25, 2009

(54) Gel Scaffolds for Tissue Engineering

(75) Inventors: Yi-You Huang, Taipei City (TW); Yi-Chieh Wu, Taipei City (TW); Ching-Hua Wang, Taipei City (TW)

Correspondence Address: WPAT, PC, 7225 BEVERLY ST., ANNANDALE, VA, 22003, US

(73) Assignee: National Taiwan University, Taipei City (TW)

(21) Appl. No.: 12/245,894

(22) Filed: Oct. 6, 2008

(30) Foreign Application Priority Data
Dec. 25, 2007 (TW) 096149833

(51) Int. Cl. A61L 27/54

(52) U.S. Cl. 424/423

(57) ABSTRACT

The present invention discloses a colloid scaffolds for tissue engineering, comprising collagen and hyaluronic acid (HA). The collagen and the hyaluronic acid (HA) are mixed to form colloidal suspension. The ratio of the collagen and the hyaluronic acid is less than or equal to 300, and the best is less than or equal to 200. The colloidal scaffolds more comprises micro-fibers or nano-fibers which are used to enhance cell attachment and increase the strength of colloid scaffolds and reduce water loss and contraction of colloid scaffolds.

### BACKGROUND OF THE INVENTION

1. Field of the Invention

The present invention is generally related to the scaffolding material, and more particularly to the colloid scaffolds for tissue engineering.

2. Description of the Prior Art

Tissue engineering is a relatively new field of basic and clinical science that is concerned, in part, with creating tissues that can augment or replace injured, defective, or diseased body parts. The approach to fabricating the tissues involves adding specific cell types to grow on a polymer scaffold having the shape of the tissue to be restored.

The reconstruction of soft tissue defects is a problem; an ideal filler material for the correction of congenital deformities, cancer defects has still to be found. Autologous mature adipose tissue has been used as free grafts for the reconstruction of soft tissue defects for more than 100 years and is still in use because of the lack of a better alternative, although the results are poor and unpredictable. The transplants are largely absorbed and replaced by fibrous tissue and oil cysts. The poor results of free fat autotransplantation are thought to be due to the low tolerance of mature fat cells to ischemia and the slow rate of revascularisation of the grafts.

Using tissue-engineering approach, cultured preadipocytes were transplanted instead of mature cells to test the hypothesis that these cells would display better survival. Preadipocytes can undergo differentiation and dedifferentiation in vitro under specific culture conditions and are a potential material for soft tissue engineering due to their ability to proliferate and differentiate into adipose tissue after transplantation. Preadipocytes need optimal biodegradable carriers with specific properties in terms of type of material and structure which allow these cells to invade and differentiate after transplantation. Mechanical stability of the carrier is important and it should not be resorbed too quickly after transplantation.

The main study of the adipose tissue engineering with collagen and hyaluronic acid (HA) is still based on porous solid scaffolds or sponge scaffolds. Although cells can penetrate the sponge scaffolds, the penetrated depth is still limited. The adipose tissue is usually confined in the surface of the scaffolds and can't distribute the whole scaffolds. The collagen scaffolds is an ideal one for cells to distribute in the scaffolds, but it still has some disadvantages. The collagen scaffolds shrink because of the distributed cells and the lower the permeability. The consequence is the innutrition caused the cells apoptosis. According to this reason, the adipose tissue engineering will develop the efficient, high biocompatible, safe and permanent engineering collagen tissue materials.

### SUMMARY OF THE INVENTION

In light of the above background about inconvenience and disadvantages, in order to fulfill the requirements of the industry, the present invention provides the colloid scaffolds for tissue engineering to solve the target that can not be achieved by the cell smear apparatus in the prior art.

One object of the present invention is using collagen and hyaluronic acid (HA) as the materials of colloid scaffolds for tissue engineering. The colloid scaffolds not only mixed with cells directly, but also prevent the contraction of the colloid scaffolds.

Another object of the present invention is to provide the injectable colloid scaffolds for tissue engineering. The advantage of injectable colloid scaffolds is improving safety, reducing invasive damage, and improving patient's acceptance.

Accordingly, the present invention discloses the colloid scaffolds for tissue engineering, comprising collagen and hyaluronic acid (HA). The collagen and the hyaluronic acid (HA) are mixed to form colloidal suspension. The ratio of the collagen and the hyaluronic acid is less than or equal to 300, and the best is less than or equal to 200.

### BRIEF DESCRIPTION OF THE DRAWINGS

FIG. 1 shows the activity and proliferation of preadipocyte under the different ratio colloid condition by the MTS assay;

FIG. 2 is shows the activity and proliferation of fibroblast under the different ratio colloid condition by the MTS assay;

FIG. 3 shows the subcutaneous injection of the hyaluronic acid-collagen colloid mixed with Preadipocytes by weighted imaging MRI T2;

FIG. 4 shows the subcutaneous injection of the hyaluronic acid-collagen colloid mixed with preadipocytes by weighted imaging MRI T1;

FIG. 5 shows the colloidal scaffolds more comprises micro-fibers or nano-fibers;

FIG. 6 shows the colloidal scaffolds more comprises the control release particles, and these particles contain drugs and hormones.

### DESCRIPTION OF THE PREFERRED EMBODIMENTS

What probed is into the invention the colloid scaffolds for tissue engineering. Detail descriptions of the structure and elements will be provided in the following in order to make the invention thoroughly understood. Obviously, the application of the invention is not confined to specific details familiar to those who are skilled in the art. On the other hand, the common structures and elements that are known to everyone are not described in details to avoid unnecessary limits of the invention. Some preferred embodiments of the present invention will now be described in greater detail in the following specification. However, it should be recognized that the present invention can be practiced in a wide range of other embodiments besides those explicitly described, that is, this invention can also be applied extensively to other embodiments, and the scope of the present invention is expressly not limited except as specified in the accompanying claims.

Tissue engineering is a relatively young field that combines engineering, clinical science, and life sciences to, in part, repair or regrow tissues. Adipose tissue has recently become a focus area for tissue engineering, encouraged by the large number of reconstructive, cosmetic, and correctional indications that could be addressed with clinically translatable adipose tissue engineering strategies. In tissue engineering approach, there are three major categories in this field: cells, scaffold, and growth factory.

The first embodiment of the invention discloses the colloid scaffolds for tissue engineering, comprising collagen and hyaluronic acid (HA). The collagen and the hyaluronic acid (HA) are mixed to form colloidal suspension. The ratio of the collagen and the hyaluronic acid is less than or equal to 300, and the best is less than or equal to 200. Therefore, the viscosity of the colloid scaffolds is about 10 to 1000 cps, and the best is about 10 to 500 cps.

The above-mentioned colloid scaffolds is injectable colloid scaffolds. Traditional solid porous scaffold is implanted into human body via surgical operation, it is inconvenient to use, and the cells cannot be distributed evenly. So, the use of injectable colloid scaffolds to replace the traditional surgical implant not only improves safety and reduces invasive damage but also improves the patient's acceptance.

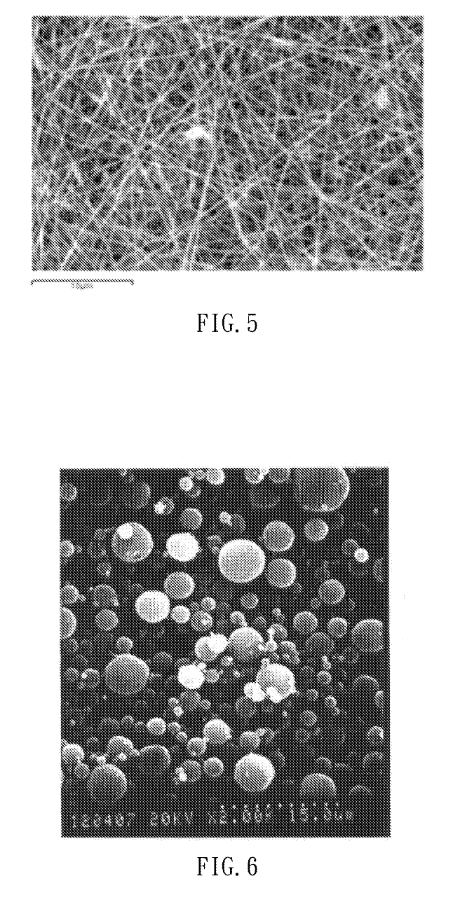

The above-mentioned colloid scaffolds is used for culturing mammalian cells, cartilage cells, mesenchymal stem cells, embryonic stem cells, hair follicle stem cells, fat cells, preadipocytes. The above-mentioned colloid scaffold is particularly suitable for culturing preadipocytes. The colloid scaffolds mix with cells evenly to form colloidal cell structure before use. As show in FIG. 5, the colloidal cell structure more comprises micro-fibers or nano-fibers which are used to enhance cell attachment and increase the strength of colloid scaffolds and reduce water loss and contraction of colloid scaffolds.

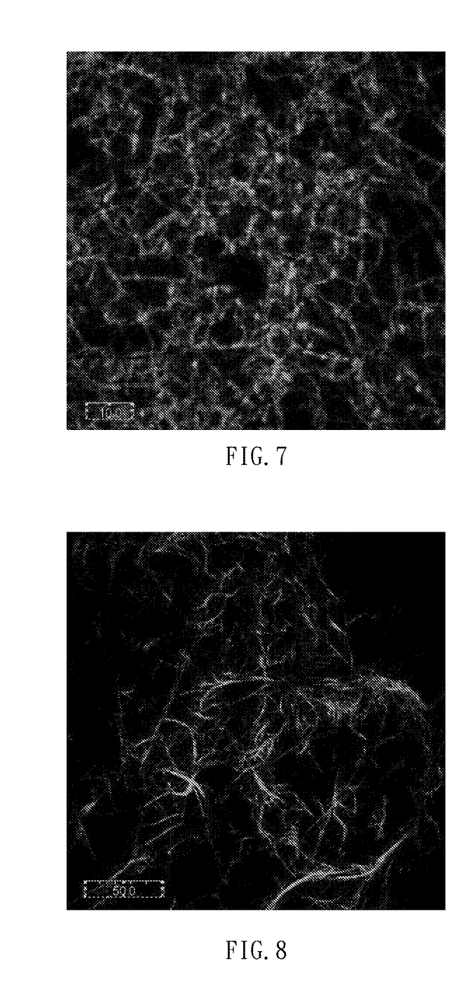

As show in FIG. 6, the colloidal cell structure more comprises the control release particles, and these particles contain drugs and hormones. This drugs and hormones can induce cell differentiation and promote cell growth. The material of the control release particles comprises one selected from the group consisting of following: insulin, dexamethasone, tri-iodothyronine, thiazolidinedione, fibroblast growth factors(FGFs), epidermal growth factor(EGF), vascular endothelial growth factor, bone morphogenetic protein(BMP), nerve growth factor (NGF), brain-derived neurotrophic factor(BDNF), and platelet-derived growth factor(PDGF), etc.

### EXAMPLE 1

This invention discloses the colloid scaffolds for tissue engineering, comprising collagen and hyaluronic acid (HA). The collagen and the hyaluronic acid (HA) are mixed to form colloidal suspension.

### I. Collagen Preparation

1. Solution preparation
A. Citric Acid buffer
   Mix 0.2M Sodium Citrate solution with o.2M Citric Acid solution and adjust the PH value to 4.5.
B. Pepsin solution
   0.5M HCl/Pepsin=20/1 (w/w)

2. Cut off the fat, muscle or some connective tissue from the pigskin completely.

3. Mince the pigskin and put it in Acetone solution. Keep stirring in room temperature for 30 minutes to remove fat. (repeat this step twice)

4. Wash the minced pigskin with double distilled water and make sure there is no water soluble or non-collagen substance residue.

5. Immerse the minced pigskin in 10% NaCl solution. Stir in 4° C. for 24 hours to make it become swollen.

6. Wash the minced pigskin with double distilled water and make sure there is no NaCl residue.

7. Immerse the minced pigskin in pH 4.5 citric acid buffer solution. Stir in 4° C. for 24 hours to make it become swollen.

8. Put the minced pigskin in pepsin solution. Stir in 4° C. for 24 hours to make it be decomposed. (pepsin/original weight of pigskin=1/10).

9. Centrifuge the sticky product solution formed by the decomposed minced pigskin in 22000 g (12650 rpm) for 1 hour in 4° C. The purpose is to separate the decomposed product from the residual tissue mass.

10. Mix the supernatant with NaCl solution (NaCl final concentration is 5% (w/w)). The white substance from salting out is the collagen product.

11. Centrifuge the collagen product solution in 22000 g for 1 hour in 4° C.

12. Dissolve the collagen with 0.5M Acetic Acid.

13. Take some dissolved collagen out and dialyze it with MWCO=50000 dialytic membrane. This step can separate the impurities out and higher the collagen purity. The rest collagen should be stocked in −20° C.

14. Sterilize the Collagen by high speed centrifugation in 14000 rpm for 30 minutes in room temperature.

15. The ⅓ top supernatant is the sterile collagen product.

### II. The Preparation of the Colloid of Collagen and Hyaluronic Acid Mixture

Preparation Method A:
1. Mix Hyaluronic Acid with double distilled water completely. Sterilize the solution by the high speed centrifugation (14000 rpm for 30 minutes in room temperature). Harvest the ⅓ top supernatant to be the sample for the further experiments.
2. Mix the proportional Hyaluronic Acid colloid with the collagen colloid and make it evenly for the further experiments.

Preparation Method B:
1. Mix the proportional Hyaluronic Acid powder with the collagen colloid directly and make it evenly for the further experiments.

### III. The Primary Culture of the Preadipocyte and the Induced Differentiation.

1. Sacrifice the male rat by CO2. Shave the rat abdominal hair and then soak the whole rat in 70% Alcohol for 10 minutes sterilization.

2. Get the adipose tissue surround epididymis and kidney of the male rat in sterile laminar flow hood and then immerse the sample in 37° C. shipping medium.

3. Mince the adipose tissue first and then mix with Collagenase(60000 U Collagenase/10 g tissue). Put the mixture in shaking water sink for 100 rpm, 37° C., 60-90 minutes.

4. Use the sterile organza(70 pores/pore size 192 um) to filter the decomposed adipose tissue solution for cleaning the un-decomposed tissue mass.

5. Centrifuge the decomposed adipose tissue solution in 1000 rpm for 10 minutes.

6. Reconstitute the cell pellet with medium and centrifuge it again in 1000 rpm, 10 minutes. Repeat this step twice for washing the cell pellet.

7. Reconstitute the cell pellet with RBC dissolved buffer and then wait for 10 minutes to complete the hemolysis. Afterward, centrifuge the solution in 1000 rpm, 10 minutes.

8. Remove the supernatant and reconstitute the cell pellet with growth medium(DMEM/F12 medium with 10% FBS).

9. Use the sterile organza(150 pores/pore size 95.5 um) to filter out the blood capillary debris and the bigger tissue mass.

10. Count the cell number (preadipocyte culture density:1.5*105 cells/ml)

11. Gently distribute the cells and make it even in the flask. Incubate the cells at 37° C. in 5% CO2. Change the medium every 2˜3 days.

12. Keep incubating for 2 days after the cells get at confluence. Wash the flask with PBS then change the medium with the differentiated initiation medium for inducing the preadipocytes differentiate.

13. After incubate with the differentiated initiation medium for 2˜3 days, wash the flask with PBS then change the medium with the maintainess medium. Change the medium every 2˜3 days.

IV. Compare the Different Growth of the Preadipocytes with the Fibroblast in Different Ratio Collagen.

1. Prepare three colloids with different ratio of collagen and hyaluronic acid in 96 well plates. The ratio is listed below:

TABLE 1
Three colloid scaffolds with different ratio of collagen/hyaluric
acid.

| | Recipe | | |
|---|---|---|---|
| | (collagen:hyaluronic acid) | | |
| | (w/w) | | |
| | A | B | C |
| | 200:0 | 200:1 | 200:2 |
| 0.1 mg/ml hyaluronic acid | 0 ml | 0.715 ml | 1.055 ml |
| 11.15 mg/ml collagen | 2 ml | 1.285 ml | 0.945 ml |
| Total volume | 2 ml | 2 ml | 2 ml |

2. Separate the Wistar Rat's preadipocytes and the fibroblast from primary culture into 3 different groups. Culture with the different ratio colloid.

3. Use MTS assay to evaluate the cell growing condition in day1, day 3, day 7 and day 10.

### V. Animal Experiment

First animal experiment is to inject the pure collagen colloid and observe by MRI to make the rough evaluation. The second animal experiment is to inject the collagen colloid with hyaluronic acid to compare the result with different ratio colloid.
1. Pure collagen mix with the preadipocytes to do the subcutaneous injection.
   (1) The day before preparing the cell colloid, feed 50% of the preadipocytes with the iron oxide agent for 6 hours:
      a. Remove the old medium and wash with PBS twice.
      b. Add the growth medium with iron oxide agents. The final iron oxide agent concentration should be 2 ug/ml.
      c. After incubate 6 hours later, remove the old medium and wash with PBS twice.
      d. Add the fresh medium and keep incubating.
   (2) Harvest the preadipocytes (including 2 groups: the cells w/or w/o iron oxide agent) at the day to execute the animal experiment. Tune the cell number equal to each other.
   (3) Centrifuge the medium with cells. Afterward, re-suspend both group cell pellet with 300 ul growth medium and 100 ul Fetal Bovine Serum to become the 400 ul cell liquid.
   (4) Mix both cell liquid with 1 ml collagen colloid to become cell colloid.
   (5) Anesthesia Wistar Rat with Forane® and then shave the dorsal hair of rat.
   (6) Inject the cell colloid with 26G syringe to the dorsal site of rat. The left side is injected the cell colloid with iron oxide agent, the right side is in the opposite.
   (7) Apply the MRI detection right after the injection.
   (8) Afterward, do the MRI detection periodically to observe the location and shape of the cell colloid.
2. The collagen with different ratio of hyaluronic acid mix with the preadipocytes to do the subcutaneous injection.
   (1) The day before preparing the cell colloid, feed the preadipocytes with the iron oxide agent for 6 hours. (The iron oxide agent concentration should be 2 ug/ml)
   (2) Harvest the preadipocytes at the day to execute the animal experiment and tune the cell number to 3×10⁷.
   (3) Centrifuge the medium with cells. Afterward, re-suspend the cell pellet with 375 ul growth medium and 125 ul Fetal Bovine Serum to become the 500 ul cell liquid.
   (4) Separate the cell liquid to 3 groups and mix with the collagen and hyaluronic acid to prepare three different ratio cell colloids. The recipe as the chapter 2.7. (collagen/hyaluronic acid=200/0, 200/1, 200/2 (w/w))
   (5) Inject the cell colloid with 26G syringe to the dorsal site of Wistar rat. The volume of the injection is 0.5 ml with 10⁷ cells.
   (6) Apply the MRI detection right after the injection. Afterward, do the MRI detection periodically to observe the location and shape of the cell colloid, or whether it differentiates to the adipose tissue.

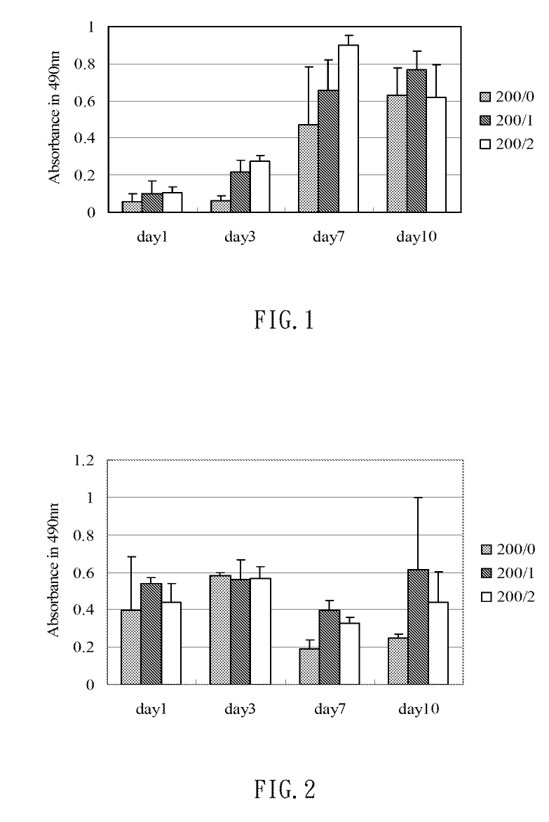

Use the in vivo cell colloid culture to evaluate the growth effect of the preadipocytes with the pure collagen or the collagen with hyaluronic acid. In this experiment, we used the same condition to culture the fibroblast as well. MTS assay can evaluate the cell activity and proliferation under the different ratio colloid condition. The result demonstrated that the collagen colloid is a good scaffold for preadipocytes. As the time pass by, cell proliferated obviously, see FIG. 1. The colloid with some hyaluronic acid was better than the pure collagen, the result in day3, day7 were most evidently, and the group with collagen/hyaluronic acid=200/2 (w/w) proliferated mostly. That means adding hyaluronic acid definitely help the preadipocytes grow in the collagen colloid, and the more the better. In the other hand, the same colloid condition didn't enhance the proliferation of the fibroblast, see FIG. 2. The cell activity of fibroblast dropped down in each group at day7, although fibroblast proliferated at day10 but was limited. Compare the culture condition with two different cell, we know that collagen/hyaluronic acid enhance the proliferation greatly of the preadipocytes. It's adaptive for culturing the preadipocytes, not also maintain the cell survival number but also keep it growing. It is with high potential of applying on adipose tissue engineer.

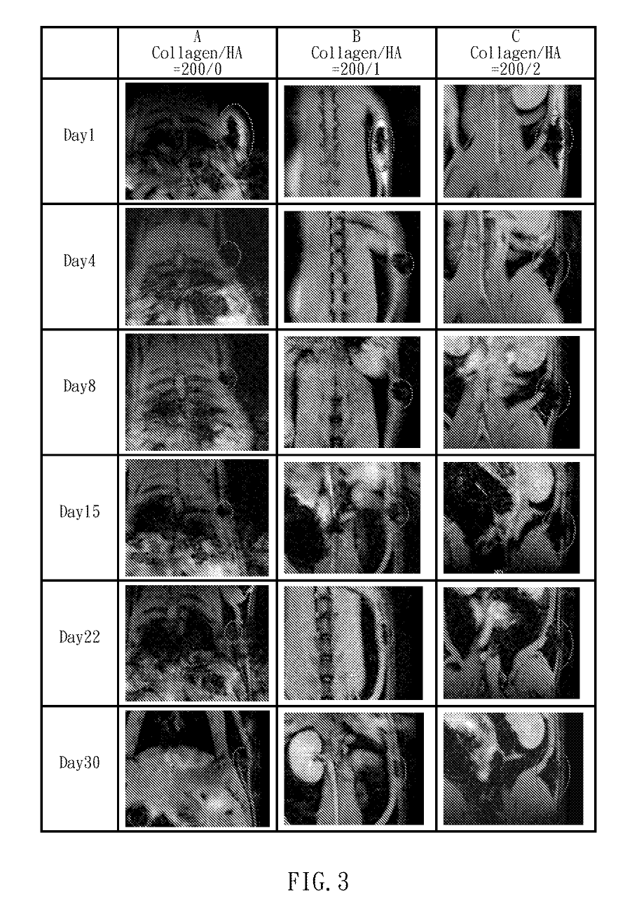

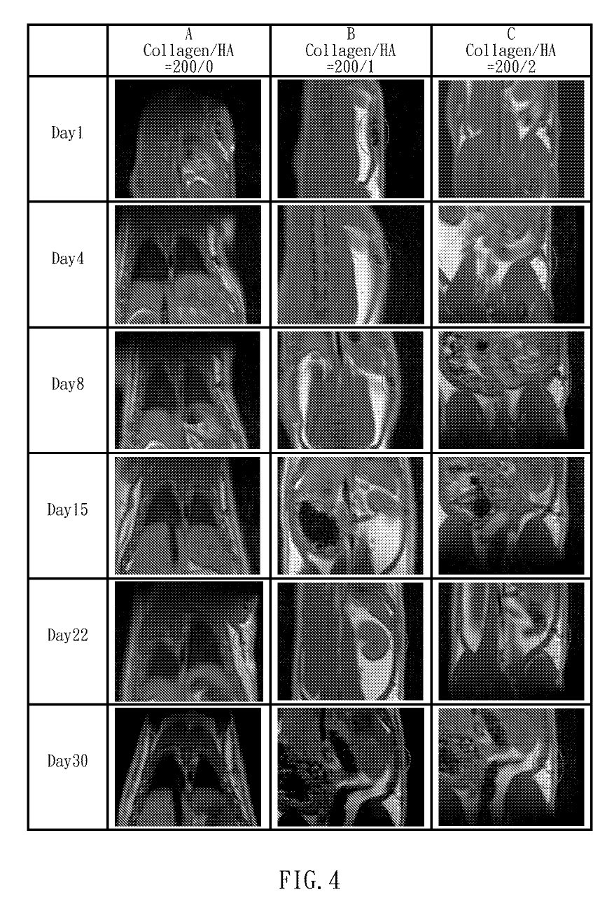

In animal experiment, we evaluated with the subcutaneous injection. We found that adding hyaluronic acid in collagen can prevent the colloid shrink (contract). The best effective recipe is collagen/hyaluronic acid=200/2 (w/w). Based on the weighted imaging MRI (FIG. 3), the colloid can keep the same original size and shape around 1 month. From the T1 weighted image (FIG. 4), cells did differentiate steadily at day15.

The in vivo or in vitro study all demonstrate that the preadipocytes can proliferate and differentiate greatly in the mixture with collagen and hyaluronic acid. Besides, the cell colloid can maintain the size and shape in the very long period of time. It would be very potential of the repair tissue in the soft tissue deficiency repair engineer.

Obviously many modifications and variations are possible in light of the above teachings. It is therefore to be understood that within the scope of the appended claims the present invention can be practiced otherwise than as specifically described herein. Although specific embodiments have been illustrated and described herein, it is obvious to those skilled in the art that many modifications of the present invention may be made without departing from what is intended to be limited solely by the appended claims.

What is claimed is:

1. A colloid scaffolds for tissue engineering, comprising collagen and hyaluronic acid (HA) wherein said collagen and said hyaluronic acid (HA) are mixed to form colloidal suspension.

2. The colloid scaffolds for tissue engineering according to claim 1, wherein said colloid scaffolds is injectable colloid scaffolds.

3. The colloid scaffolds for tissue engineering according to claim 1, wherein said tissue engineering is adipose tissue engineering.

4. The colloid scaffolds for tissue engineering according to claim 1, wherein the ratio of said collagen and said hyaluronic acid is less than or equal to 300.

5. The colloid scaffolds for tissue engineering according to claim 1, wherein the ratio of said collagen and said hyaluronic acid is less than or equal to 200.

6. The colloid scaffolds for tissue engineering according to claim 1, wherein the viscosity of said colloid scaffolds is about 10 to 1000 cps.

7. The colloid scaffolds for tissue engineering according to claim 1, wherein the viscosity of said colloid scaffolds is about 10 to 500 cps.

8. The colloid scaffolds for tissue engineering according to claim 1, wherein said colloid scaffolds is used for culturing cells or stem cells, and said colloid scaffolds mix with said cells or said stem cells evenly.

9. The colloid scaffolds for tissue engineering according to claim 1, wherein said colloid scaffolds is used for culturing fat cells, and said colloid scaffolds mix with said fat cells evenly.

10. The colloid scaffolds for tissue engineering according to claim 1, wherein said colloid scaffolds is used for culturing preadipocytes, and said colloid scaffolds mix with said preadipocytes evenly.

11. The colloid scaffolds for tissue engineering according to claim 1, wherein said colloid scaffolds mix with cells evenly to form colloidal cell structure before use.

12. The colloid scaffolds for tissue engineering according to claim 11, wherein said colloidal cell structure more comprises micro-fibers or nano-fibers wherein said micro-fibers or said nano-fibers is used to enhance cell attachment and increase the strength of colloid scaffolds and reduce water loss and contraction of colloid scaffolds.

13. The colloid scaffolds for tissue engineering according to claim 11, wherein said colloidal cell structure more comprises the control release particles, and the material of said control release particles comprises one selected from the group consisting of following: insulin, dexamethasone, tri-iodothyronine, thiazolidinedione, fibroblast growth factors(FGFs), epidermal growth factor(EGF), vascular endothelial growth factor, bone morphogenetic protein(BMP), nerve growth factor (NGF), brain-derived neurotrophic factor(BDNF), and platelet-derived growth factor(PDGF).
